(19) United States
(12) Patent Application Publication
(10) Pub. No.: US 2018/0282712 A1
(43) Pub. Date: Oct. 4, 2018

(54) TRANSGLUTAMINASE VARIANTS HAVING INCREASED SPECIFIC ACTIVITY

(71) Applicant: BRISTOL-MYERS SQUIBB COMPANY, Princeton, NJ (US)

(72) Inventor: Chetana RAO-NAIK, Walnut Creek, CA (US)

(21) Appl. No.: 15/763,962

(22) PCT Filed: Sep. 30, 2016
(86) PCT No.: PCT/US2016/054590

§ 371 Date: Mar. 28, 2018

Related U.S. Application Data
(60) Provisional application No. 62/236,282, filed on Oct. 2, 2015.

(51) Int. Cl. C12N 9/10; C07K 16/00; A61K 47/68; A61K 51/10

(52) U.S. Cl. CPC C12N 9/1044; C12Y 203/02013; C07K 16/00; C07K 2317/41; A61K 51/10; C07K 2319/00; A61K 47/6835

(57) ABSTRACT

A variant transglutaminase having increased specific activity compared to wild-type Streptomyces mobaraensis transglutaminase can be used for conjugating proteins.

### CROSS-REFERENCE TO RELATED APPLICATIONS

This application claims the benefit under 35 U.S.C. § 119(e) of U.S. Provisional Application No. 62/236,282, filed Oct. 2, 2015; the disclosure of which is incorporated herein by reference.

### SEQUENCE LISTING

Incorporated herein by reference in its entirety is a Sequence Listing named “12614WOPCT_ST25.txt,” comprising SEQ ID NO:1 through SEQ ID NO:14, which include nucleic acid and/or amino acid sequences disclosed herein. The Sequence Listing has been submitted herewith in ASCII text format via EFS-Web, and thus constitutes both the paper and computer readable form thereof. The Sequence Listing was first created using PatentIn 3.5 on Sep. 24, 2015, and is approximately 27 KB in size.

### BACKGROUND OF THE INVENTION

Transglutaminase is an enzyme that can form an amide bond between the carboxamide side chain of a glutamine (the amine acceptor) in a first protein and the ε-amino group of a lysine (the amine donor) in a second protein, in a transamidation reaction. Thus, it can join two proteins together, or conjugate them. Transglutaminase has found many applications in biotechnology and in the food processing industry, where it has earned the sobriquet “meat glue.”

The most commonly used transglutaminase is bacterial transglutaminase from Streptomyces mobaraensis, having an amino acid sequence according to SEQ ID NO:1 and referred to hereinafter as BTG. Specificity-wise, it is selective regarding the glutamine residue, requiring that it be located in a flexible part of a protein loop and flanked by particular amino acids. Conversely, BTG is permissive regarding the lysine residue: it evenaccepts an amino group from a non-protein source, such an alkyleneamino compound, as a lysine ε-amino surrogate. See Fontana et al. 2008.

Tagami et al. 2009 and Yokoyama et al. 2010 have studied the effect of mutations on the specific activity of BTG against the dipeptide N-carbobenzoxy-L-glutaminylglycine (and also ovalbumin in the case of Tagami et al. 2009) as an amine acceptor. The substitutions they made and their effects on specific activity are summarized in Table 1 and Tables 2-4 thereof, respectively.

Another use for BTG is in making antibody conjugates. In such use, the antibody is conjugated—i.e., covalently attached—to another chemical moiety. The moiety can be, for instance, another protein, a radioisotope, an assay agent (e.g., biotin or a fluorescent label), or a drug. A particularly preferred conjugate is an antibody-drug conjugate (ADC), in which the antibody is conjugated to a drug (also variously referred to as the warhead or payload).

Antibodies of the IgG isotype have many glutamines—nine or more in the heavy chain constant region alone, the exact number depending on isotype. However, none of them are BTG-reactive in a native antibody—that is, they are not transamidated by transglutaminase—and some modification of the antibody is necessary to induce reactivity. Normally, an antibody is glycosylated at asparagine 297 (N297) of the heavy chain (N-linked glycosylation). Jeger 2009 and Jeger et al. 2010 disclosed that deglycosylation of the antibody, either by eliminating the glycosylation site through an N297A substitution or post-translation enzymatic deglycosylation by an enzyme such as PNGase F (peptide-N-glycosidase F), renders nearby glutamine 295 (Q295) transglutaminase-reactive. (References to amino acid positions in an antibody constant region employ numbering per the EU index as set forth in Kabat et al., “Sequences of proteins of immunological interest,” 5th ed., Pub. No. 91-3242, U.S. Dept. Health & Human Services, NIH, Bethesda, Md., 1991; hereinafter “Kabat.”) They further showed that an N297Q substitution not only eliminates glycosylation, but also introduces a second glutamine residue, at position 297, that is an amine acceptor. Thus, simple deglycosylation generates two transglutaminase-reactive glutamine residues per antibody (one per heavy chain, at Q295), while an N297Q substitution will generate four transglutaminase-reactive glutamine residues (two per heavy chain, at Q295 and Q297).

In summary, to conjugate an antibody using BTG, either the enzyme or the antibody needs to be modified. In one approach, the structure of an antibody is modified to make it transglutaminase-reactive. In addition to the modifications disclosed by Jeger 2009 and Jeger et al. 2010, discussed above, a glutamine-containing peptide, or “tag,” can be added to an antibody. See Dorywalska et al. 2015; Pons et al. 2013 and Rao-Naik 2015. The tag can be a glutamine inserted or substituted into the antibody—that is, a single amino acid insertion or substitution—or the tag can be a glutamine-containing polypeptide inserted at the N-terminus, middle, or C-terminus of an antibody chain, commonly but not necessarily the heavy chain.

In another approach, BTG is mutated to make it capable of using Q295 as an amine receptor, even where N297 is glycosylated. See Rao-Naik et al., U.S. Provisional Application Ser. No. 62/236,724, filed Oct. 2, 2015. Others have also investigated altering the glutamine specificity of BTG by altering its amino acid sequence. Working with human growth hormone (hGH), Norskov-Lauritsen et al. 2009 found that the selectivity of BTG for Gln-40 compared to Gln141 in hGH can be improved by replacing up to three basic or acidic amino acid residues with other basic or acidic amino acids. Working with a different organism, Streptoverticillium ladakanum, Hu et al. 2009, 2010a, and 2010b reported that the selectivity of its transglutaminase for Gln-141 could be increased by modifying its amino acid sequence at certain positions or by adding residues to its N-terminus.

Other transglutaminase disclosures, generally relating to the labeling or modification of proteins (including antibodies), are: Bregeon 2014, Bregeon et al. 2013 and 2014, Chen et al. 2005, Fischer et al. 2014, Hu et al. 2015, Kamiya et al. 2011, Lin et al. 2006, Mero et al. 2009, Mindt et al. 2008, Sato 2002, Sato et al. 2001, Schlibi et al. 2007, and Sugimura et al. 2007.

Full citations for the documents cited herein by first author or inventor and year are listed at the end of this specification.

### BRIEF SUMMARY OF THE INVENTION

It is desirable that a transglutaminase have high specific activity, by which is meant the activity of a transglutaminase in transamidating a reference amine acceptor and a reference amine donor, compared to that of another transglutaminase. If a high specific activity transglutaminase is used in a process, whether in biotechnology or food processing, less enzyme needs to be used and the transamidation step can be completed in a shorter time. With less enzyme present in the conjugation reaction mixture, purification is easier.

The present invention provides a transglutaminase variant having increased specific activity, compared to wild type transglutaminase (SEQ ID NO: 1). Thus, this invention provides a variant transglutaminase polypeptide comprising an amino acid sequence that is at least 90% identical (preferably at least 95% identical and more preferably 100% identical) to SEQ ID NO:1, with the proviso that the variant transglutaminase polypeptide has a V65I and a Y75F substitution, i.e., a double substitution where valine 65 is replaced by an isoleucine and tyrosine 75 is replaced by a phenylalanine.

The present invention also provides a method of making an antibody conjugate, comprising:
(a) mixing an antibody having a transglutaminase-reactive glutamine with an amine donor compound comprising an primary amine and a moiety selected from the group consisting of a protein, a radioisotope, an assay agent, and a drug, in the presence of a variant transglutaminase comprising an amino acid sequence that is at least 90% identical (preferably at least 95% identical and more preferably 100% identical) to SEQ ID NO:1, with the proviso that the variant transglutaminase has a V65I and a Y75F amino acid substitution; and
(b) allowing the variant transglutaminase to catalyze the formation of an amide bond between the side chain carboxamide of the transglutaminase-reactive glutamine and the primary amine of the amine donor compound, thereby making the antibody conjugate.

In another aspect, the present invention provides another method of making an antibody conjugate, comprising:
(a) mixing an antibody having a transglutaminase-reactive glutamine with a first compound, which first compound is an amine donor compound having a primary amine and a first reactive functional group, in the presence of a variant transglutaminase comprising an amino acid sequence that is at least 90% identical (preferably at least 95% identical and more preferably 100% identical) to SEQ ID NO:1, with the proviso that the variant transglutaminase has a V65I and a Y75F amino acid substitution;
(b) allowing the variant transglutaminase to catalyze the formation of an amide bond between the side chain carboxamide of the transglutaminase-reactive glutamine and the primary amine of the first compound, to make an adduct of the antibody and the first compound;
(c) contacting the adduct with a second compound having a second reactive functional group and a moiety selected from the group consisting of a protein, a radioisotope, an assay agent, and a drug; the second reactive functional group being capable of reacting with the first reactive functional group to form a covalent bond therebetween; and
(d) allowing the first and second reactive functional groups to react and form a covalent bond therebetween, thereby making the antibody conjugate.

Where moiety (in the first compound or second compound, as the case may be) is a protein, the resultant conjugate is a fusion protein. Where the moiety is a radioisotope, the resultant conjugate can be used for radiation therapy. The moiety can be an assay agent such as a fluorescent label or a ligand like biotin, in which case the conjugate can be used for diagnostic or analytical applications. Preferably, the moiety is a drug, in which case the product is an antibody-drug conjugate, which can be used in medical treatments, especially the treatment of cancer.

### BRIEF DESCRIPTION OF THE DRAWING(S)

FIG. 1 shows schematically the BTG mediated preparation of an ADC, via two processes respectively referred to as the one-step and the two-step process.

### DETAILED DESCRIPTION OF THE INVENTION

One transglutaminase variant of this invention, designated M1, has a double mutation (V65I and Y75F), relative to the sequence of the wild-type S. mobaraensis transglutaminase (SEQ ID NO:1). The amino acid sequence of variant M1 is shown in SEQ ID NO:4. As shown hereinbelow, variant M1 has a specific activity almost one-and-a-half time greater than wild-type BTG.

This application also discloses for comparison another variant, designated M2, which has a double mutation (Y62H and Y75F), relative to the sequence of the wild-type S. mobaraensis transglutaminase (SEQ ID NO:1). The amino acid sequence of variant M2 is shown in SEQ ID NO:5.

This application further discloses for comparison yet another variant, designated M4, which has a single mutation (Q74A), relative to the sequence of the wild-type S. mobaraensis transglutaminase (SEQ ID NO:1). The amino acid sequence of variant M4 is shown in SEQ ID NO:6.

This application further discloses for comparison yet another variant, designated M5, which has a triple mutation (W69A, Q74A, and Y75F), relative to the sequence of the wild-type S. mobaraensis transglutaminase (SEQ ID NO:1). The amino acid sequence of variant M5 is shown in SEQ ID NO:7.

Variants M1, M2, M4, and M5 can have conservative substitutions thereto, provided their respective distinctive substitutions (a) V65I/Y75F, (b) Y62H/Y75F, (c) Q74A, or (d) W69A/Q74A/Y75F, respectively, are preserved. Such conservatively modified versions of variants M1, M2, M4, and M5 are included in the scope of this invention. A “conservative modification” or “conservative substitution” means, in respect of a polypeptide, the replacement of an amino acid therein with another amino acid having a similar side chain. Families of amino acids having similar side chains are known in the art. Such families include amino acids with basic side chains (lysine, arginine, histidine), acidic side chains (aspartic acid, glutamic acid), uncharged polar side chains (asparagine, glutamine, serine, threonine, tyrosine, cysteine, tryptophan), nonpolar side chains (glycine, alanine, valine, leucine, isoleucine, proline, phenylalanine, methionine), beta-branched side chains (threonine, valine, isoleucine), small side chains (glycine, alanine, serine), chain orientation changing side chains (glycine, proline) and aromatic side chains (tyrosine, phenylalanine, tryptophan). Plural conservative substitutions/modifications may be present. Preferably, conservatively modified versions of variants M1, M2, M4, and M5 are at least 90% identical, more preferably at least 95% identical to their respective unmodified sequences.

BTG variants M1, M2, M4, and M5 may further comprise an N-terminal extension of a tetrapeptide according to SEQ ID NO:8 (FRAP).

BTG variants M1, M2, M4, and M5 may further comprise a polyhistidine peptide extension at their C-terminus, as exemplified with amino acid residues 336-441 of SEQ ID NO:3. The polyhistidine peptide is a useful tag for purification purposes and does not affect enzymatic activity. Typically, the polyhistidine peptide is 6-8 residues long.

An antibody can be endowed with a BTG-reactive glutamine by unmasking Q295 by removing glycosylation at position 297, either enzymatically with an enzyme such as PNGase F (peptidyl N-glycosidase F) or by performing a site specific substitution replacing N297 with a different amino acid. As noted above, an N297Q substitution also introduces a BTG-reactive glutamine, namely Q297. Also, a glutamine tag can be introduced to the antibody, either within the amino acid chain or by an extension at either the N-terminus or C-terminus thereof, preferably the latter. The tag is commonly but not necessarily located on the antibody heavy chain.

Antibodies that can be conjugated by the methods of this invention (assuming modification as discussed above to render them BTG-reactive) include those recognizing the following antigens: mesothelin, prostate specific membrane antigen (PSMA), CD19, CD22, CD30, CD70, B7H3, B7H4 (also known as 08E), protein tyrosine kinase 7 (PTK7), glypican-3, RG1, fucosyl-GM1, CTLA-4, and CD44. The antibody can be animal (e.g., murine), chimeric, humanized, or, preferably, human. The antibody preferably is monoclonal, especially a monoclonal human antibody. The preparation of human monoclonal antibodies against some of the aforementioned antigens is disclosed in Korman et al., U.S. Pat. No. 8,609,816 B2 (2013; B7H4, also known as 08E; in particular antibodies 2A7, 1G11, and 2F9); Rao-Naik et al., U.S. Pat. No. 8,097,703 B2 (2012; CD19; in particular antibodies 5G7, 13F1, 46E8, 21D4, 21D4a, 47G4, 27F3, and 3C10); King et al., U.S. Pat. No. 8,481,683 B2 (2013; CD22; in particular antibodies 12C5, 19A3, 16F7, and 23C6); Keler et al., U.S. Pat. No. 7,387,776 B2 (2008; CD30; in particular antibodies 5F11, 2H9, and 17G1); Terrett et al., U.S. Pat. No. 8,124,738 B2 (2012; CD70; in particular antibodies 2H5, 10B4, 8B5, 18E7, and 69A7); Korman et al., U.S. Pat. No. 6,984,720 B1 (2006; CTLA-4; in particular antibodies 10D1, 4B6, and 1E2); Vistica et al., U.S. Pat. No. 8,383,118 B2 (2013, fucosyl-GM1, in particular antibodies 5B1, 5B1a, 7D4, 7E4, 13B8, and 18D5) Korman et al., U.S. Pat. No. 8,008,449 B2 (2011; PD-1; in particular antibodies 17D8, 2D3, 4H1, 5C4, 4A11, 7D3, and 5F4); Huang et al., US 2009/0297438 A1 (2009; PSMA. in particular antibodies 1C3, 2A10, 2F5, 2C6); Cardarelli et al., U.S. Pat. No. 7,875,278 B2 (2011; PSMA; in particular antibodies 4A3, 7F12, 8C12, 8A11, 16F9, 2A10, 2C6, 2F5, and 1C3); Terrett et al., U.S. Pat. No. 8,222,375 B2 (2012; PTK7; in particular antibodies 3G8, 4D5, 12C6, 12C6a, and 7C8); Terrett et al., U.S. Pat. No. 8,680,247 B2 (2014; glypican-3; in particular antibodies 4A6, 11E7, and 16D10); Harkins et al., U.S. Pat. No. 7,335,748 B2 (2008; RG1; in particular antibodies A, B, C, and D); Terrett et al., U.S. Pat. No. 8,268,970 B2 (2012; mesothelin; in particular antibodies 3C10, 6A4, and 7B1); Xu et al., US 2010/0092484 A1 (2010; CD44; in particular antibodies 14G9.B8.B4, 2D1.A3.D12, and 1A9.A6.B9); Deshpande et al., U.S. Pat. No. 8,258,266 B2 (2012; IP10; in particular antibodies 1D4, 1E1, 2G1, 3C4, 6A5, 6A8, 7C10, 8F6, 10A12, 10A12S, and 13C4); Kuhne et al., U.S. Pat. No. 8,450,464 B2 (2013; CXCR4; in particular antibodies F7, F9, D1, and E2); and Korman et al., U.S. Pat. No. 7,943,743 B2 (2011; PD-L1; in particular antibodies 3G10, 12A4, 10A5, 5F8, 10H10, 1B12, 7H1, 11E6, 12B7, and 13G4); the disclosures of which are incorporated herein by reference.

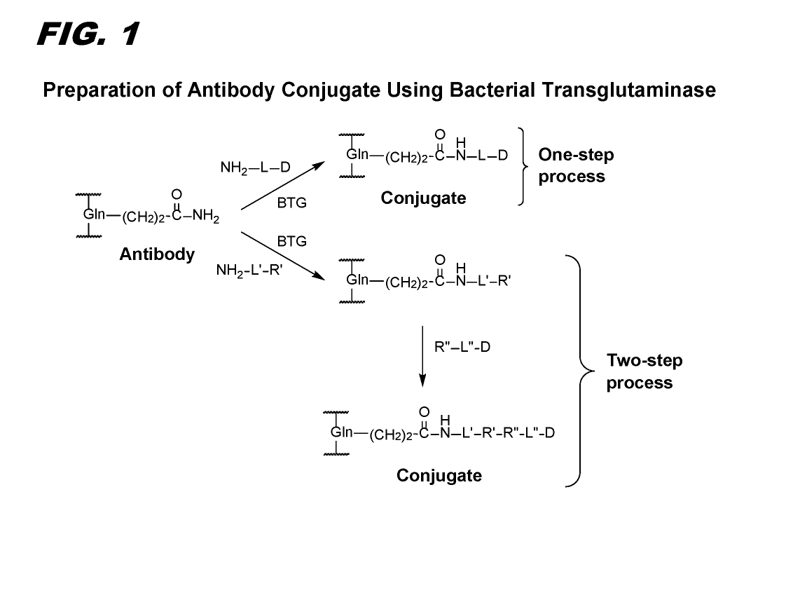

BTG-mediated preparation of an antibody conjugate can be by a one-step process or a two-step process, as illustrated schematically in FIG. 1. In the one-step process, BTG couples a BTG-reactive glutamine carboxamide on the antibody acting as the amine acceptor and an amine donor compound H2N-L-D, where L is a linker moiety and D is a protein, a radioisotope, an assay agent, or a drug, to form the conjugate directly. In the two-step process, BTG catalyzes formation an initial transamidation adduct between a BTG-reactive glutamine carboxamide acting as the amine receptor and an amine donor compound H2N-L′-R′, where L′ is a linker moiety and R′ is a first reactive functional group. Subsequently the adduct is reacted with a compound R″-L″-D, where R″ is a second reactive functional group capable of reacting with R′, L″ is a linker moiety, and D is as defined above. Sometimes, the one-step process is referred to as the enzymatic process, and the two-step process as the chemo-enzymatic process.

The amine donor, whether H₂N-L-D or H₂N-L′-R′, is often used in large excess to suppress undesired transamidation between the glutamine carboxamide and an ε-amino group of an antibody lysine. If the moiety D is expensive or difficult to obtain, the use of a large excess may be impractical. In such instances, the two-step process may be preferable.

In a preferred embodiment, amine donor compound in a one-step process is represented by formula (I):

H₂N—(CH₂)₂₋₆D (I)

where D is a drug.

More preferably, the one-step method is used to make an ADC. The amine donor compound can have a structure represented by formula (Ia):

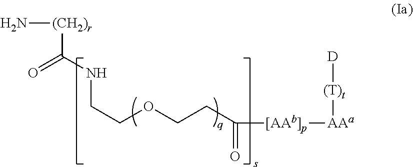

D is a drug;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
q is 1, 2, 3, 4, 5, 6, 7, 8, 9, or 10;
r is 1, 2, 3, 4, or 5; and
s is 0 or 1.

In formula (Ia), -AA^{a}-[AA^{b}]ₚ- represents a polypeptide whose length is determined by the value of p (dipeptide if p is 1, tetrapeptide if p is 3, etc.). AA^{a} is at the carboxy terminus of the polypeptide and its carboxyl group forms a peptide (amide) bond with an amine nitrogen of drug D (or self-immolating group T, if present). Conversely, the last AA^{b} is at the amino terminus of the polypeptide and its α-amino group forms a peptide bond with

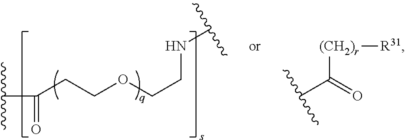

depending on whether s is 1 or 0, respectively. Preferred polypeptides -AA^{a}-[AA^{b}]ₚ- are Val-Cit, Val-Lys, Lys-Val-Ala, Asp-Val-Ala, Val-Ala, Lys-Val-Cit, Ala-Val-Cit, Val-Gly, Val-Gln, and Asp-Val-Cit, written in the conventional N-to-C direction, as in H₂N-Val-Cit-CO₂H). More preferably, the polypeptide is Val-Cit, Val-Lys, or Val-Ala. Preferably, a polypeptide -AA^{a}-[AA^{b}]ₚ- is cleavable by an enzyme found inside the target (cancer) cell, for example a cathepsin and especially cathepsin B.

If the subscript s is 1, drug-linker (Ia) contains a poly(ethylene glycol) (PEG) group, which can advantageously improve the solubility of drug-linker (Ia), facilitating conjugation to the antibody—a step that is performed in aqueous media. Also, a PEG group can serve as a spacer between the antibody and the peptide -AA^{a}-[AA^{b}]ₚ-, so that the bulk of the antibody does not sterically interfere with action of a peptide-cleaving enzyme.

As indicated by the subscript t equals 0 or 1, a self-immolating group T is optionally present. A self-immolating group is one such that cleavage from AA^{a} or AA^{b}, as the case may be, initiates a reaction sequence resulting in the self-immolating group disbonding itself from drug D and freeing the latter to exert its therapeutic function. When present, the self-immolating group T preferably is ap-aminobenzyl oxycarbonyl (PABC) group, whose structure is shown below, with an asterisk (*) denoting the end of the PABC bonded to an amine nitrogen of drug D and a wavy line ( [symbol] ) denoting the end bonded to the polypeptide -AA^{a}-[AA^{b}]ₚ-.

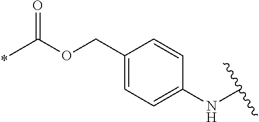 [SMILES: *Nc1ccc(COC(C)=O)cc1]

Another self-immolating group that can be used is a substituted thiazole, as disclosed in Feng, U.S. Pat. No. 7,375,078 B2 (2008).

In a two-step conjugation, many combinations of groups R′ and R″ can be used. Suitable combinations of R′ and R″ (or, vice-versa, R″ and R′) include:
(a) a maleimide group and a sulfhydryl group, to form a Michael addition adduct, as in

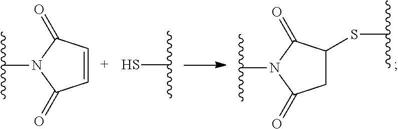

(b) a dibenzocyclooctyne group and an azide group, to form a cycloaddition product via “click” chemistry, as in

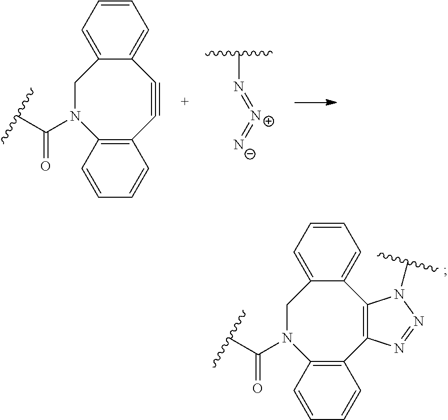

(c) an N-hydroxysuccinimide ester and an amine, to form an amide, as in

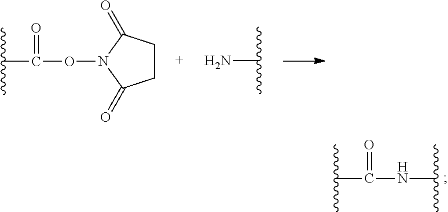

and
(d) an aldehyde or ketone (where “alkyl” below preferably is C₁₋₃ alkyl) and a hydroxylamine, to form an oxime, as in

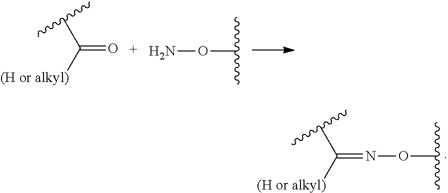

Thus, R′ can be selected from

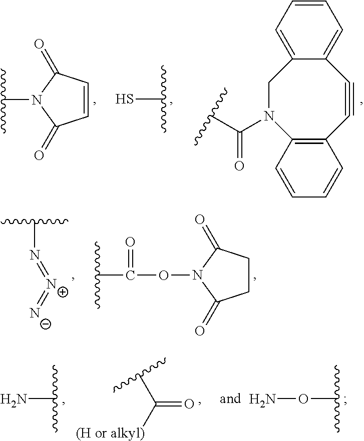

while, reciprocally, R″ can be selected from

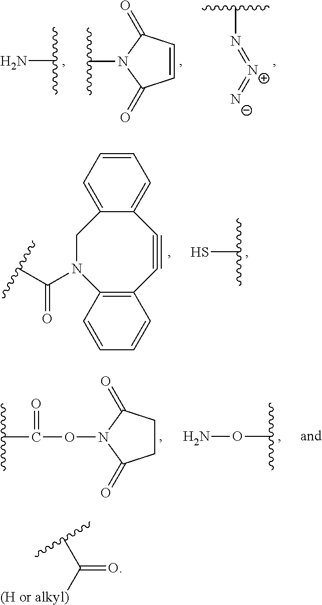

A suitable amine donor compound H₂N-L′-R′ for the two-step process is depicted in formula (II)

H₂N—(CH₂)₂₋₈—R′ (II)

where R′ is as defined above and preferably is

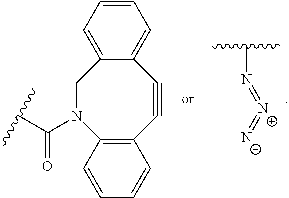

A corresponding suitable compound R″-L″-D is shown in formula (III)

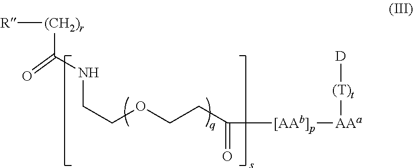

where R″ is as defined above and preferably is

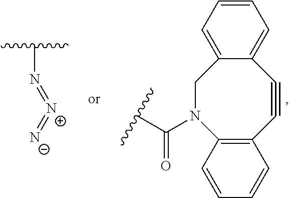

and r, q, s, AA^{b}, p, AA^{a}, T, t, and D are as defined above in respect of formula (la).

In the instance where the conjugate is an ADC intended for use in cancer treatment, drug D preferably is a cytotoxic drug that causes death of the targeted cancer cell. Cytotoxic drugs that can be used in ADCs include the following types of compounds and their analogs and derivatives:
(a) enediynes such as calicheamicin (see, e.g., Lee et al., J. Am. Chem. Soc. 1987, 109, 3464 and 3466) and uncialamycin (see, e.g., Davies et al., WO 2007/038868 A2 (2007); Chowdari et al., U.S. Pat. No. 8,709,431 B2 (2012); and Nicolaou et al., WO 2015/023879 A1 (2015));
(b) tubulysins (see, e.g., Domling et al., U.S. Pat. No. 7,778,814 B2 (2010); Cheng et al., U.S. Pat. No. 8,394,922 B2 (2013); and Cong et al., U.S. Pat. No. 8,980,824 B2 (2015));
(c) DNA alkylators such as analogs of CC-1065 and duocarmycin (see, e.g., Boger, U.S. Pat. No. 6,5458,530 B1 (2003); Sufi et al., U.S. Pat. No. 8,461,117 B2 (2013); and Zhang et al., U.S. Pat. No. 8,852,599 B2 (2014));
(d) epothilones (see, e.g., Vite et al., US 2007/0275904 A1 (2007) and U.S. RE42930 E (2011));
(e) auristatins (see, e.g., Senter et al., U.S. Pat. No. 6,844,869 B2 (2005) and Doronina et al., U.S. Pat. No. 7,498,298 B2 (2009));
(f) pyrrolobezodiazepine (PBD) dimers (see, e.g., Howard et al., US 2013/0059800 A1 (2013); US 2013/0028919 A1 (2013); and WO 2013/041606 A1 (2013)); and
(g) maytansinoids such as DM1 and DM4 (see, e.g., Chari et al., U.S. Pat. No. 5,208,020 (1993) and Amphlett et al., U.S. Pat. No. 7,374,762 B2 (2008)).

Preferably, the drug is a DNA alkylator, tubulysin, auristatin, pyrrolobenzodiazepine, enediyne, or maytansinoid compound. Specific examples include:

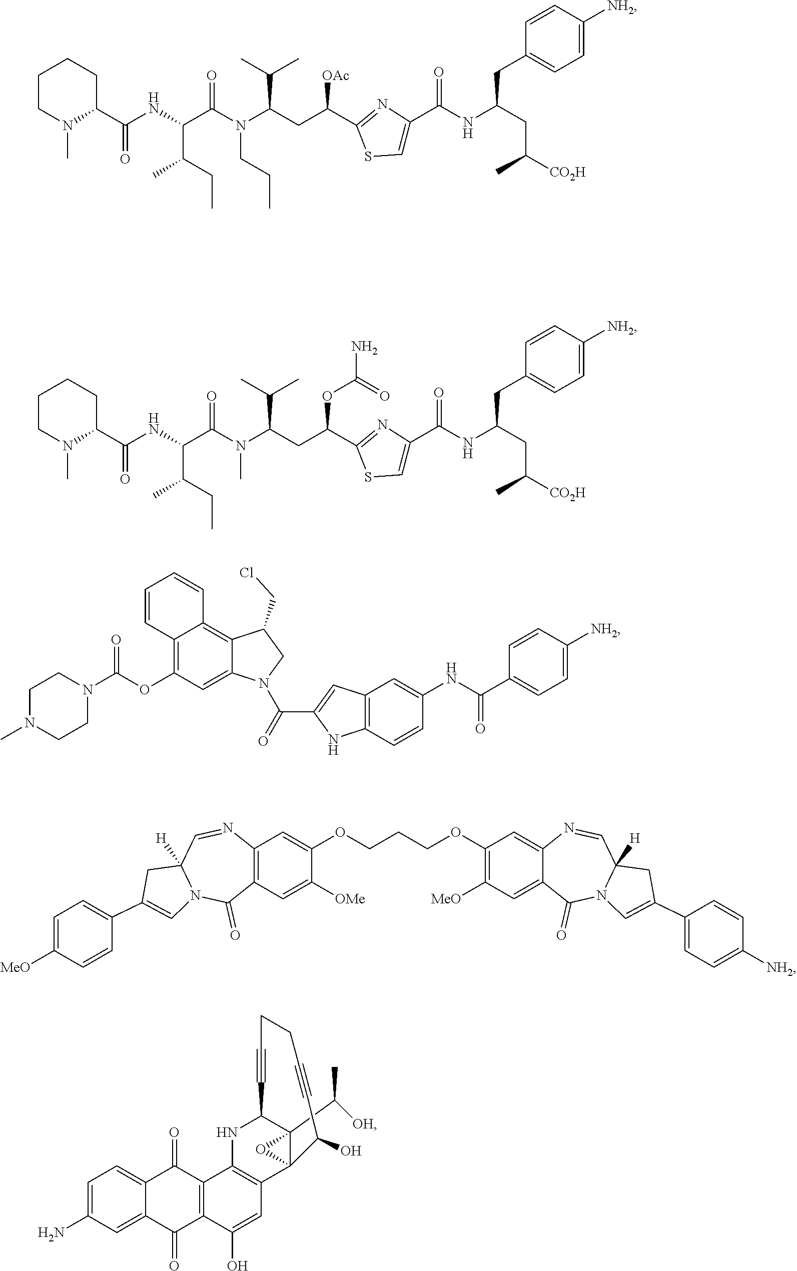

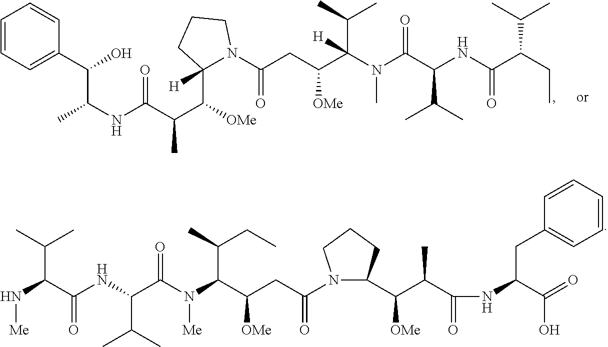

The functional group at which conjugation is effected is the amine (—NH₂) group in the case of the first five drugs above and the methyl amine (—NHMe) group in the case of the last two drugs.

The aforementioned drug moieties can be used in ADCs made by either the one-step or two-step process.

The foregoing references, in addition to disclosing the drug moieties proper, also disclose drug-linker constructs according to formulae (Ia) or (III), or which can be readily adapted to make such drug-linker compounds, mutatis mutandis. Particularly pertinent disclosures relating to the preparation of drug-linker compounds are found in Chowdari et al., U.S. Pat. No. 8,709,431 B2 (2012); Cheng et al., U.S. Pat. No. 8,394,922 B2 (2013); Cong et al., U.S. Pat. No. 8,980,824 B2 (2015); Sufi et al., U.S. Pat. No. 8,461,117 B2 (2013); and Zhang et al., U.S. Pat. No. 8,852,599 B2 (2014). While these references may relate to specific drug moieties, those skilled in the art will appreciate that the principles of making drug-linker compounds there are applicable to other types of drugs, mutatis mutandis.

A glutamine in an antibody is BTG-reactive (synonymously, transglutaminase-reactive) if its carboxamide side chain acts as an amine acceptor for S. mobaraensis transglutaminase (SEQ ID NO:1), using hydroxylamine as the amine donor.

In one embodiment, the antibody having a BTG-reactive glutamine is an IgG antibody aglycosylated at position 297. As disclosed by Jeger 2009 and Jeger et al. 2010, the disclosures of which are incorporated herein by reference, aglycosylation can be achieved by by treatment with an enzyme such as PNGase F (peptide-N-glycosidase F) or by an N297A amino acid substitution, which eliminates the Asn 297 glycosylation site. In either case, the result is that nearby Gln 295 (Q295) is made BTG-reactive.

In another embodiment, the antibody having a BTG-reactive glutamine is an IgG antibody having an N297Q amino acid substitution, which, as disclosed in Jeger 2009 and Jeger et al. 2010, generates two BTG-reactive glutamines (Q295 and Q297).

In yet another embodiment, the antibody having a BTG-reactive glutamine has glutamine-containing peptide inserted therein. The peptide can be inserted at the N-terminus, the C-terminus, or in the middle of the antibody. The See Dorywalska et al. 2015; Pons et al. 2013 and Rao-Naik 2015. The peptide can have from one to ten amino acids, preferably from four to eight amino acids.

The practice of this invention can be further understood by reference to the following examples, which are provided by way of illustration and not of limitation.

### Example 1

The amino acid sequence of S. mobaraensis transglutaminase (BTG) is provided in SEQ ID NO:1. For generating the mutants of this invention, BTG was produced recombinantly by expression in E. coli, initially producing a proenzyme according SEQ ID NO:2. Activation by cleavage of an N-terminal peptide by dispase yielded recombinant BTG according to SEQ ID NO:3, which contained an FRAP tetrapeptide at the N-terminus and a polyhistidine tail at the C-terminus (amino acids 1-4 and 336-441 of SEQ ID NO:3, respectively). The core part of SEQ ID NO:3 (amino acids 5-335) was identical to SEQ ID NO:1. This recombinant BTG had the same activity as wild-type BTG. The preparation of recombinant BTG used herein is described in detail below.

Bacterial transglutaminase from S. mobaraensis was expressed in E. coli as a proenzyme with a C-terminal His-tag. Bacterial cell pellets expressing the proenzyme were collected and treated as follows: The pellet was weighed while frozen. For each 1 g of pellet, 2 mL of BPER II reagent, 0.5 mg/mL lysozyme, 0.5 U/mL BENZONASE® endonuclease (EMD Millipore), and one protease inhibitor tablet were added to re-suspend the pellet. After the re-suspension was homogenous, it was transferred to centrifuge tubes and centrifuged at 27000×g for 15 min. The supernatant was decanted into a separate container and extra re-suspension buffer was added to the pellet for further re-suspension and centrifuged at 27000×g for 15 minutes. This process was repeated twice and the collected supernatant fractions were pooled. The pooled supernatant fractions were filtered through a 0.2 μm filter before loading onto a column for purification.

A 5 mL HisTrap® Excel column was equilibrated with 50 mM tris-HCl, 300 mM NaCl, 2 mM CaCl₂, 1 mM glutathione, pH 8.0 for 10 CV. The extracted protein (˜40 mL) was loaded onto the column. The column was then washed with equilibration buffer (˜20 column volumes). The equilibration buffer with 1.3 mg/mL of dispase enzymewas then used to wash the column until baseline increased as an indication that dispase has been equilibrated within the column. The column was removed from the instrument and incubated at 37° C. for 1 h. Post incubation, the column was washed with equilibration buffer (without dispase) until baseline was reached. The activated protein was eluted with 35% Buffer B (50 mM Tris-HCl, 300 mM NaCl, 500 mM Imidazole pH 8.0).

The collected peak fractions from the elution were pooled and dialyzed overnight with 50 mM Na acetate, 500 mM NaCl pH 5.5. After dialysis, the final material was filtered through a 0.2 μm filter, aliquoted and stored at −80° C.

The Microbial Transglutaminase kit from Zedira was used to measure the specific activity of BTG and the variant transglutaminases of this invention. The kit uses N-carbobenzoxy-L-glutaminylglycine (Z-Gln-Gly or CBZ-Gln-Gly) as the amine acceptor substrate and hydroxylamine as amine donor. In the presence of transglutaminae, the hydoxylamine is incorporated to form Z-glutamylhydroxamate-glycine which develops a colored complex with iron (III) detectable at 525 nm.

### Example 2

Two different inserts were constructed for optimizing expression of bacterial transglutaminase in E. coli. One insert was used for periplasmic expression and the other for inclusion body expression. The inserts were codon optimized and include a C-terminal (His)6 tag.

For periplasmic expression, the M1 transglutaminase insert (1234 base pairs, SEQ ID NO:13) was amplified by PCR using primers zg67,899 (SEQ ID NO:9) and zg67,900 (SEQ ID NO:10). The transglutaminase plasmid was made by homologously recombining pCHAN51 acceptor vector (derived in-house) and PCR amplified transglutaminase M1 donor PCR fragment. The resulting construct, designated pSDH779, was transformed into competent E. coli DH10B for protein expression.

For inclusion body expression, the M1 transglutaminase insert (1238 base pairs, SEQ ID NO:14) was amplified by PCR primers zg67,903 (SEQ ID NO:11) and zg67,904 (SEQ ID NO:12). The transglutaminase plasmid was made by homologously recombining pTAP238 acceptor vector (derived in-house) and PCR amplified transglutaminase M1 donor PCR fragment. The resulting plasmid was designated pSDH784 and transformed into competent E. coli DH10B for protein expression.

Mutants M2, M4, and M5 were analogously prepared.

### Example 3

The specific activities of variant M1, a BTG control (unmutated) and other comparative variants are provided in Table 1. The activities were obtained using the Zedira kit referenced above and the substrate pair Z-Gln-Gly and hydroxylamine.

TABLE 1
Specific Activity of Transglutaminases

Specific Activity

| | Concentration | | Relative to |
|---|---|---|---|
| Transglutaminase | (mg/mL) | U/mg | control |
| Control | 0.04 | 8.8 | — |
| Variant M1 | 0.09 | 12.5 | 1.4 |
| Variant M2 | 0.09 | 8.5 | 0.97 |
| Variant M4 | 0.09 | 8.5 | 0.97 |
| Variant M5 | 0.05 | 6.8 | 0.77 |
| V65I mutant^{(a)} | — | — | 1.3 |
| Y75F mutant^{(a)} | — | — | 1.5 |

^{(a)}As reported in Yokoyama et al. 2010, Table 1.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

### REFERENCES

Full citations for the following references cited in abbreviated fashion by first author (or inventor) and date earlier in this specification are provided below. Each of these references is incorporated herein by reference for all purposes.
Bregeon et al., US 2013/0189287 A1 (2013).
Bregeon, WO 2014/202773 A1 (2014).
Bregeon et al., WO 2014/202775 A1 (2014).
Chen et al., US 2005/0136491 A1 (2005).
Dennler et al., Bioconjug. Chem. 2014, 25, 569.
Dorywalska et al., Bioconjug. Chem. 2015, 26, 650.
Fischer et al., WO 2014/072482 A1 (2014).
Fontana et al., Adv. Drug Deliv. Rev. 2008, 60, 13.
Hu et al., US 2009/0318349 A1 (2009).
Hu et al., US 2010/0087371 A1 (2010) [2010a].
Hu et al., US 2010/0099610 A1 (2010) [2010b].
Hu et al., WO 2015/191883 A1 (2015).
Innate Pharma, “A New Site Specific Antibody Conjugation Using Bacterial Transglutaminase,” presentation at ADC Summit, San Fransisco, Calif., Oct. 15, 2013.
Jeger, Doctoral Thesis, ETH Zurich, “Site-Specific Conjugation of Tumour-Targeting Antibodies Using Transglutaminase” (2009).
Jeger et al., Angew. Chem. Int. Ed. 2010, 49, 9995.
Kamiya et al., US 2011/0184147 A1 (2011).
Lhospice et al., Mol. Pharmaceutics 2015, 12, 1863.
Lin et al., J. Am. Chem. Soc. 2006, 128, 4542-4543.
Mero et al., Bioconjug. Chem. 2009, 384-389.
Mindt et al., Bioconjug. Chem. 2008, 19, 271.
Norskov-Lauritsen et al., US 2009/0117640 A1 (2009).
Pons et al., US 2013/0230543 A1 (2013).
Rao-Naik, U.S. Provisional application Ser. No. 62/130,673, filed Mar. 7, 2015.
Sato et al., U.S. Pat. No. 6,322,996 B1 (2001).
Sato, Adv. Drug Deliv. Rev. 2002, 54, 487.
Schibli et al., US 2007/0184537 A1 (2007).
Schrama et al., Nature Rev. Drug Disc. 2006, 5, 147.
Strop et al., Chemistry & Biology 2013, 20, 161.
Sugimura et al., J. Biotechnol. 2007, 131, 121.
Tagami et al., Protein Engineering Design Selecttion 2009, 22 (12), 747.
Yokoyama et al., Appl. Microbiol. Biotechnol. 2010, 87, 2087.

### Table of Sequences

TABLE 2
Sequence Summary

| SEQ ID NO: | SEQUENCE DESCRIPTION |
|---|---|
| 1 | S. mobaraensis BTG a.a. |
| 2 | Recombinant S. mobaraensis BTG proenzyme a.a. |
| 3 | Activated recombinant S. mobaraensis BTG a.a. |
| 4 | Variant M1 a.a. |
| 5 | Variant M2 a.a. |
| 6 | Variant M4 a.a. |
| 7 | Variant M5 a.a. |
| 8 | N-terminal tetrapeptide a.a. |
| 9 | Primer zg67,899 n.t. |
| 10 | Primer zg67,900 n.t. |
| 11 | Primer zg67,903 n.t. |
| 12 | Primer zg67,904 n.t. |
| 13 | M1 amplicon, periplasmic, n.t. |
| 14 | M1 amplicon, inclusion body, n.t. |

What is claimed is:

1. A variant transglutaminase comprising an amino acid sequence that is at least 90% identical to SEQ ID NO:1, with the proviso that the variant transglutaminase has a V65I and a Y75F amino acid substitution.

2. A variant transglutaminase according to claim 1, comprising the amino acid sequence of SEQ ID NO:4.

3. A method of making an antibody conjugate, comprising:
(a) mixing an antibody having a transglutaminase-reactive glutamine with an amine donor compound comprising an primary amine and a moiety selected from the group consisting of a protein, a radioisotope, an assay agent, and a drug, in the presence of a variant transglutaminase comprising an amino acid sequence that is at least 90% identical (preferably at least 95% identical and more preferably 100% identical) to SEQ ID NO:1, with the proviso that the variant transglutaminase has a V65I and a Y75F amino acid substitution; and
(b) allowing the variant transglutaminase to catalyze the formation of an amide bond between the side chain carboxamide of the transglutaminase-reactive glutamine and the primary amine of the amine donor compound, thereby making the antibody conjugate.

4. A method according to claim 3, wherein the variant transglutaminase comprises the amino acid sequence of SEQ ID NO:4.

5. A method according to claim 3, wherein the antibody is an IgG antibody aglycosylated at position 297.

6. A method according to claim 4, wherein the antibody has a glutamine-containing peptide inserted therein.

7. A method according to claim 4, wherein the amine donor compound has a structure represented by formula (I)
H₂N—(CH₂)₂₋₆D (I)
where D is a drug.

8. A method according to claim 4, wherein the amine donor compound has a structure represented by formula (Ia) 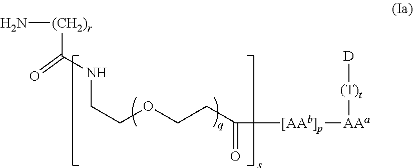
wherein
D is a drug, preferably a DNA alkylator, tubulysin, auristatin, pyrrolobenzodiazepine, enediyne, or maytansinoid compound;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
q is 1, 2, 3, 4, 5, 6, 7, 8, 9, or 10;
r is 1, 2, 3, 4, or 5; and
s is 0 or 1.

9. A method of making an antibody conjugate, comprising:
(a) mixing an antibody having a transglutaminase-reactive glutamine with a first compound, which first compound is an amine donor compound having a primary amine and a first reactive functional group, in the presence of a variant transglutaminase comprising an amino acid sequence that is at least 90% identical (preferably at least 95% identical and more preferably 100% identical) to SEQ ID NO:1, with the proviso that the variant transglutaminase has a V65I and a Y75F amino acid substitution;
(b) allowing the variant transglutaminase to catalyze the formation of an amide bond between the side chain carboxamide of the transglutaminase-reactive glutamine and the primary amine of the first compound, to make an adduct of the antibody and the first compound;
(c) contacting the adduct with a second compound having a second reactive functional group and a moiety selected from the group consisting of a protein, a radioisotope, an assay agent, and a drug; the second reactive functional group being capable of reacting with the first reactive functional group to form a covalent bond therebetween; and
(d) allowing the first and second reactive functional groups to react and form a covalent bond therebetween, thereby making the antibody conjugate.

10. A method according to claim 9, wherein the variant transglutaminase comprises the amino acid sequence of SEQ ID NO:4.

11. A method according to claim 9, wherein the antibody is an IgG antibody aglycosylated at position 297.

12. A method according to claim 9, wherein the antibody has a glutamine-containing peptide inserted therein.

13. A method according to claim 9, wherein the first compound has a structure represented by formula (II)
H₂N—(CH₂)₂₋₈—R′ (II)
wherein
R′ is selected from 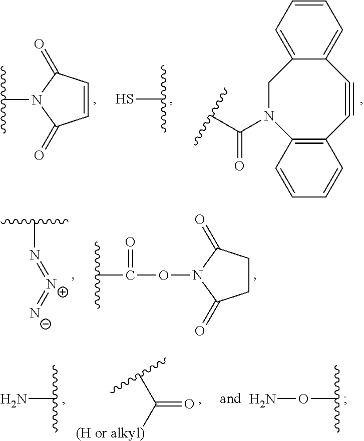
and the second compound has a structure represented by formula (III) 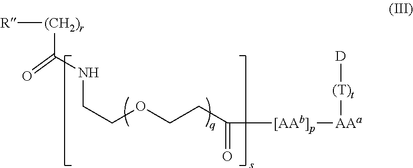
wherein
R″ is selected from 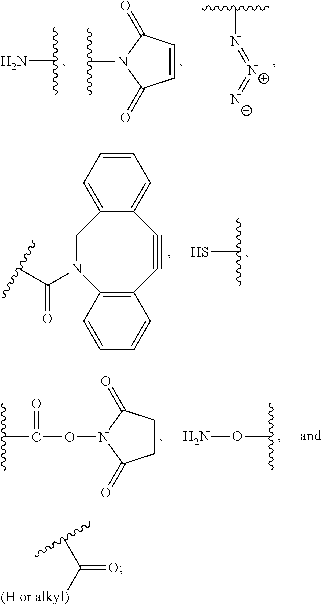
D is a drug, preferably a a DNA alkylator, tubulysin, auristatin, pyrrolobenzodiazepine, enediyne, or maytansinoid compound;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
q is 1, 2, 3, 4, 5, 6, 7, 8, 9, or 10;
r is 1, 2, 3, 4, or 5; and
s is 0 or 1.

14. A method according to claim 13, wherein R′ is 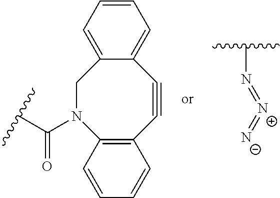
and R″ is [chemical structure]


SEQUENCE LISTING (14 sequences)

SEQ ID NO: 1 - protein, 331 aa, Streptomyces mobaraensis
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 2 - protein, 382 aa, Artificial sequence
  Recombinant transglutaminase proenzyme
DNGAGEETKSYAETYRLTADDVANINALNESAPAASSAGPSFRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPHHHHHH

SEQ ID NO: 3 - protein, 341 aa, Artificial sequence
  Activated recombinant transglutaminase
  MISC_FEATURE (1)..(4): N-Terminal tetrapeptide
  MISC_FEATURE (336)..(341): C-Terminal polyhistidine
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPHHHHHH

SEQ ID NO: 4 - protein, 331 aa, Artificial sequence
  Variant M1
  VARIANT (65)..(65): V65I substitution
  VARIANT (75)..(75): Y75Fsubstitution
DSDDRVTPPAEPLDRMPDPYRPSYGRAETVVNNYIRKWQQVYSHRDGRKQQMTEEQREWLSYGCIGVTWVNSGQFPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 5 - protein, 331 aa, Artificial sequence
  Variant M2
  VARIANT (62)..(62): Y62H substitution
  VARIANT (75)..(75): Y75F substitution
DSDDRVTPPAEPLDRMPDPYRPSYGRAETVVNNYIRKWQQVYSHRDGRKQQMTEEQREWLSFGCVGVTWVNSGQFPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 6 - protein, 331 aa, Artificial sequence
  Variant M4
  VARIANT (74)..(74): Q74A substitution
DSDDRVTPPAEPLDRMPDPYRPSYGRAETVVNNYIRKWQQVYSHRDGRKQQMTEEQREWLSYGCVGVTWVNSGAYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 7 - protein, 331 aa, Artificial sequence
  Variant M5
  VARIANT (69)..(69): W69A substitution
  VARIANT (74)..(74): Q74A substitution
  VARIANT (75)..(75): Y75F substitution
DSDDRVTPPAEPLDRMPDPYRPSYGRAETVVNNYIRKWQQVYSHRDGRKQQMTEEQREWLSYGCVGVTAVNSGAFPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 8 - protein, 4 aa, Artificial sequence
  N-terminal tetrapeptide
FRAP

SEQ ID NO: 9 - DNA, 62 bp, Artificial Sequence
  Primer zg67.899
gcgctggctggtttagttttagcgtttagcgcatcggcggataacggcgcgggcgaagaaac

SEQ ID NO: 10 - DNA, 91 bp, Artificial Sequence
  Primer zg67,900
tctgtatcaggctgaaaatcttatctcatccgccaaaacattagtgatggtgatggtgatgtgaacccggccagccctgtttcactttatc

SEQ ID NO: 11 - DNA, 68 bp, Artificial Sequence
  Primer zg67,903
tagaaataattttgtttaactttaagaaggagatatatatatggataatggtgccggtgaagaaacca

SEQ ID NO: 12 - DNA, 92 bp, Artificial Sequence
  Primer zg67,904
tctgtatcaggctgaaaatcttatctcatccgccaaaacattagtgatggtgatggtgatgtgaacccggccaaccctgtttaactttatcc

SEQ ID NO: 13 - DNA, 1234 bp, Artificial Sequence
  M1 amplicon, periplasmic
gcgctggctggtttagttttagcgtttagcgcatcggcggataacggcgcgggcgaagaaaccaaaagctatgcggaaacctatcgcctgaccgcggatgatgtggcgaacattaacgcgctgaacgaaagcgcgccggcggcgagcagcgcgggcccgagctttcgcgcgccggatagcgatgatcgcgtgaccccgccggcggaaccgctggatcgcatgccggatccgtatcgcccgagctatggccgcgcggaaaccgtggtgaacaactatattcgcaaatggcagcaggtgtatagccatcgcgatggccgcaaacagcagatgaccgaagaacagcgcgaatggctgagctatggctgcattggcgtgacctgggtgaacagcggccagtttccgaccaaccgcctggcgtttgcgagctttgatgaagatcgctttaaaaacgaactgaaaaacggccgcccgcgcagcggcgaaacccgcgcggaatttgaaggccgcgtggcgaaagaaagctttgatgaagaaaaaggctttcagcgcgcgcgcgaagtggcgagcgtgatgaaccgcgcgctggaaaacgcgcatgatgaaagcgcgtatctggataacctgaaaaaagaactggcgaacggcaacgatgcgctgcgcaacgaagatgcgcgcagcccgttttatagcgcgctgcgcaacaccccgagctttaaagaacgcaacggcggcaaccatgatccgagccgcatgaaagcggtgatttatagcaaacatttttggagcggccaggatcgcagcagcagcgcggataaacgcaaatatggcgatccggatgcgtttcgcccggcgccgggcaccggcctggtggatatgagccgcgatcgcaacattccgcgcagcccgaccagcccgggcgaaggctttgtgaactttgattatggctggtttggcgcgcagaccgaagcggatgcggataaaaccgtgtggacccatggcaaccattatcatgcgccgaacggcagcctgggcgcgatgcatgtgtatgaaagcaaatttcgcaactggagcgaaggctatagcgattttgatcgcggcgcgtatgtgattacctttattccgaaaagctggaacaccgcgccggataaagtgaaacagggctggccgggttcacatcaccatcaccatcactaatgttttggcggatgagataagattttcagcctgatacaga

SEQ ID NO: 14 - DNA, 1238 bp, Artificial Sequence
  M1 amplicon, inclusion body
tagaaataattttgtttaactttaagaaggagatatatatatggataatggtgccggtgaagaaaccaaaagctatgcagaaacctatcgtctgaccgcagatgatgttgcaaacattaatgcactgaatgaaagcgcaccggcagcaagcagcgcaggtccgagctttcgtgcaccggatagtgatgatcgtgttacccctccggcagaaccgctggatcgtatgccggatccgtatcgtccgagctatggtcgtgccgaaaccgttgttaataactatattcgtaaatggcagcaggtctatagccatcgtgatggtcgtaaacagcagatgaccgaagaacagcgtgaatggctgagttatggttgtattggtgttacctgggttaatagcggtcagtttccgaccaatcgtctggcatttgcaagctttgatgaagatcgctttaaaaacgagctgaaaaatggtcgtccgcgtagcggtgaaacccgtgcagaatttgaaggtcgtgttgcaaaagaatccttcgatgaagaaaaaggttttcagcgtgcccgtgaagttgcaagcgttatgaatcgtgcactggaaaatgcccatgatgaaagtgcatatctggataacctgaaaaaagaactggccaatggtaatgatgcactgcgtaatgaagatgcacgtagcccgttttatagcgcactgcgcaataccccgagctttaaagaacgtaatggtggtaatcatgatccgagccgtatgaaagcagtgatctatagcaaacatttttggagcggtcaggatcgtagcagcagtgcagataaacgtaaatatggtgatccggatgcatttcgtccggcaccgggtacaggtctggttgatatgagccgtgatcgtaatattccgcgtagtccgaccagtccgggtgaaggttttgttaattttgattatggttggtttggcgcacagaccgaagcagatgccgataaaaccgtttggacccatggcaatcattatcatgcaccgaatggtagcctgggtgcaatgcatgtttatgaaagtaaatttcgcaattggagcgagggctatagcgattttgatcgtggtgcatatgtgattacctttattccgaaaagctggaataccgctccggataaagttaaacagggttggccgggttcacatcaccatcaccatcactaatgttttggcggatgagataagattttcagcctgatacaga